(19) United States
(12) Patent Application Publication
(10) Pub. No.: US 2020/0179290 A1
(43) Pub. Date: Jun. 11, 2020

(54) AMORPHOUS POWDER COMPRISING AN ANGIOTENSIN RECEPTOR BLOCKER AND A NEUTRAL ENDOPEPTIDASE INHIBITOR

(71) Applicant: Quimica Sintetica, S.A., Barcelona (ES)

(72) Inventors: Giuseppe Barreca, Montevecchia (IT); Giampiero Ventimiglia, Francavilla Fontana (IT); Sonja Bellomi, Novara (IT)

(73) Assignee: Quimica Sintetica, S.A., Barcelona (ES)

(21) Appl. No.: 16/784,716

(22) Filed: Feb. 7, 2020

Related U.S. Application Data
(62) Division of application No. 15/745,297, filed on Jan. 16, 2018.

(30) Foreign Application Priority Data
Jul. 17, 2015 (EP) 15382367.9

(51) Int. Cl. A61K 9/19; C07C 233/47; C07D 257/04; A61K 31/41; A61K 31/216; A61K 9/16; A61P 9/04; A61P 9/12

(52) U.S. Cl. CPC A61K 9/19; C07C 233/47; C07D 257/04; A61P 9/12; A61K 31/216; A61K 9/1682; A61P 9/04; A61K 31/41

(57) ABSTRACT

Several methods for the preparation of an amorphous powder that includes a 1:1 stoichiometric mixture of the trisodium salts of Valsartan and Sacubitril are described, as well as the resulting amorphous powder, pharmaceutical compositions containing it, and their use in the treatment of essential hypertension and/or cardiac failure.

### CROSS-REFERENCE TO RELATED APPLICATIONS

This application is a divisional of U.S. patent application Ser. No. 15/745,297, filed Jan. 16, 2018, which itself is a U.S. National Stage application of PCT International Patent Application Serial No. PCT/EP2016/066504, filed Jul. 12, 2016, which itself claims the benefit of European Patent Application Serial No. 15382367.9, filed Jul. 17, 2015. The disclosure of each of these applications is incorporated herein by reference in its entirety.

### FIELD OF THE INVENTION

The present invention relates to an amorphous powder comprising a 1:1 stoichiometric mixture of an angiotensin receptor blocker and a neutral endopeptidase inhibitor, to pharmaceutical compositions comprising said powder as well as to several processes for obtaining the same.

### STATE OF THE ART

Angiotensin II is a hormone that causes blood vessel to constrict. This, in turn can result in high blood pressure and strain on the heart. It is known that angiotensin II interacts with specific receptors on the surface of target cells. Two receptor subtypes for angiotensin II, namely AT1 and AT2, have been identified so far. In recent times, great efforts have been made to identify substances that bind to the AT1 receptor. Angiotensin receptor blockers (ARBs, angiotensin II antagonists) are now known to prevent angiotensin II from binding to its receptors in the walls of blood vessels, thereby resulting in lower blood pressure. Because of the inhibition of the AT1 receptor, such antagonists can be used, therefore, as anti-hypertensives or for the treatment of congestive heart failure, among other indications.

Neutral endopeptidase (NEP) is a zinc-containing metalloprotease that cleaves a variety of peptide substrates on the amino side of hydrophobic residues. Substrates for this enzyme include, but are not limited to, atrial natriuretic peptide (ANP, also known as ANF), brain natriuretic peptide (BNP), met- and leu-enkephalin, bradykinin, neurokinin A, endothelin-1 and substance P. ANP is a potent vasorelaxant and natriuretic agent.

Infusion of ANP in normal subjects resulted in a reproducible, marked enhancement of natriuresis and diuresis, including increases in fractional excretion of sodium, urinary flow rate and glomerular filtration rate. However, ANP has a short half-life in circulation, and NEP in kidney cortex membranes has been shown to be the major enzyme responsible for degrading this peptide. Thus, inhibitors of NEP (neutral endopeptidase inhibitors, NEPi) should increase plasma levels of ANP and, hence, are expected to induce natriuretic and diuretic effects.

While substances, such as angiotensin receptor blockers and neutral endopeptidase inhibitors may be useful in the control of hypertension, essential hypertension is a polygenic disease and is not always controlled adequately by monotherapy. Approximately 333 million adults in economically developed countries and about 65 million Americans (1 in 3 adults) had high blood pressure in 2000. Prolonged and uncontrolled hypertensive vascular disease ultimately leads to a variety of pathological changes in target organs, such as the heart and kidney. Sustained hypertension can lead as well to an increased occurrence of stroke.

A supramolecular complex of two active agents with different mechanisms of action, namely an angiotensin receptor antagonist and a neutral endopeptidase inhibitor dual-acting compound useful for the treatment of patients with various cardiovascular and/or renal diseases has been first disclosed in the International patent application WO 2007/056546 A1.

According to the description of WO 2007/056546 A1, the angiotensin receptor blocker is preferably Valsartan (depicted below) (also known as ((S)—N-valeryl-N-{[2′-(1H-tetrazole-5-yl)-biphenyl-4-yl]-methyl}-valine), while the neutral endopeptidase inhibitor is preferably Sacubitril (depicted below) (i.e. (2R,4S)-5-biphenyl-4-yl-5-(3-carboxy-propionylamino)-2-methyl-pentanoic acid ethyl ester).

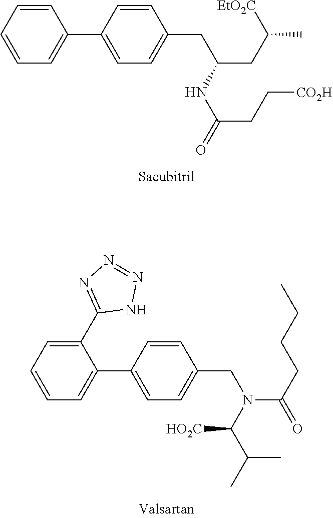 [SMILES: CCOC(=O)[C@H](C)C[C@@H](Cc1ccc(-c2ccccc2)cc1)NC(=O)CCC(=O)O ; CCCCC(=O)N(Cc1ccc(-c2ccccc2-c2nnn[nH]2)cc1)[C@H](C(=O)O)C(C)C]

A particularly useful therapeutic agent is the supramolecular complex, trisodium [3-((1S,3R)-1-biphenyl-4-yl-methyl-3-ethoxycarbonyl-1-butylcarbamoyl)propionate-(S)-3′-methyl-2′-(pentanoyl{2″-(tetrazol-5-ylate)biphenyl-4′-yl-methyl}amino)butyrate]-hemi-pentahydrate, generally referred to as LCZ-696, abbreviation that will be used in the remainder of the description and in the claims.

The formulation of dual acting compounds such as supramolecular complexes is not trivial since typical formulation techniques may have a negative effect on the drug substance leading to e.g. dissociation of the components of the dual acting compound. In general, one should avoid exposing the therapeutic agent during the formulation to moisture, excessive heat and/or high shear forces.

This may pose a number of formulation issues and difficulties, which need to be addressed.

The same patent application discloses, in example 1, that a glassy solid comprising a mixture of the sodium salts of Valsartan and Sacubitril can be prepared by evaporating at 35° C. a water/acetone mixture comprising said components. It is however worth noting that, in large scale preparations, it is not reasonable to distil the solvent under normal or reduced pressure to obtain dry solids, both for quality or safety purposes.

An object of the present invention is, therefore, to provide an amorphous powder containing a 1:1 stoichiometric mixture of an angiotensin receptor blocker and a neutral endopeptidase inhibitor, as well as pharmaceutical compositions comprising the same, which are chemically and physically stable in the amorphous state upon storage under stress conditions and that are obtainable in an easy and reliable manner, e.g. in large scale preparations.

### SUMMARY OF THE INVENTION

These objects are achieved with the present invention, which in its first aspect relates to an amorphous powder comprising a 1:1 stoichiometric mixture of the trisodium salts of Valsartan and Sacubitril and having a water content of at maximum 4% by weight. Said amorphous powder is characterized by an XRPD profile substantially as illustrated by FIG. 1.

In a second aspect thereof, the present invention relates to several methods for the preparation of the amorphous powder described above.

A first embodiment of the second aspect of the invention consists in a method for the preparation of the amorphous powder described before including the following steps:
a) dissolving LCZ-696 in water, or in a mixture comprising water and a water miscible solvent;
b) freeze-drying the solution obtained in step a); and
c) optionally, when the resulting solid contains an amount of water greater than 4% by weight, drying it.

In a possible variant of this embodiment, a further step a′) is carried out between steps a) and b), consisting in distilling off the water miscible solvent and, if necessary, dilute the mass with water to obtain a solution.

A second embodiment of this aspect of the invention consists in a method for the preparation of said amorphous powder comprising the following steps:
d) dissolving LCZ-696 in water, a water miscible solvent or a mixture thereof;
e) spray-drying the solution obtained in step d); and
f) optionally, when the resulting solid contains an amount of water greater than 4% by weight, drying it.

A third embodiment of this aspect of the invention consists in a method for the preparation of said amorphous powder including the following steps:
g) dispersing a 1:1 stoichiometric mixture of Valsartan and Sacubitril in water, a water miscible solvent or a mixture thereof;
h) adding sodium hydroxide in a ratio of 3:1 mole/mole with respect to the 1:1 stoichiometric mixture of Valsartan and Sacubitril;
i) freeze-drying the solution obtained in step h); and
j) optionally, when the resulting solid contains an amount of water greater than 4% by weight, drying it.

In a possible variant of this embodiment, a further step h′) is carried out between steps h) and i), consisting in adjusting the pH of the mass obtained in step h) to the value resulting from the dispersion of LCZ-696 in the solvent or solvents mixture used to perform steps g) and h); moreover, depending on the operating conditions (as described below), this third embodiment can optionally comprise a further step h″), carried out between steps h) and i), comprising distilling off the water miscible solvent and, if necessary, dilute the mass with water to obtain a solution. Steps h′) and h″), if carried out both, can be performed in any order.

A fourth embodiment of this aspect of the invention comprises a method for the preparation of said amorphous powder including the following steps:

k) dispersing a 1:1 stoichiometric mixture of Valsartan and Sacubitril in water, a water miscible solvent or a mixture thereof;
l) adding sodium hydroxide in a ratio of 3:1 mole/mole with respect to the 1:1 stoichiometric mixture of Valsartan and Sacubitril;
m) spray-drying the solution obtained in step l); and
n) optionally, when the resulting solid contains an amount of water greater than 4% by weight, drying it.

In a possible variant of this embodiment, a further step l′) is carried out between steps l) and m), consisting in adjusting the pH of the mass obtained in step l) to the value resulting from dispersing LCZ-696 in the solvent or solvents mixture used to perform steps k) and l).

In a third aspect thereof, the present invention relates to pharmaceutical compositions comprising said amorphous powder and at least one pharmaceutically acceptable carrier, as well as its use in the treatment of essential hypertension and/or cardiac failure.

### BRIEF DESCRIPTION OF THE FIGURE

FIG. 1 shows a XRPD spectrum of the amorphous powder.

### DETAILED DESCRIPTION OF THE INVENTION

All terms used in this application, unless otherwise specified, are to be understood in their ordinary meaning as known in the technical field. Other more specific definitions of certain terms used in this application are listed below and are intended to be applied uniformly to the entire application, unless indicated otherwise.

The term “about” includes the range of experimental error, which can normally occur when performing a measurement.

The term “mass” defines the combination of substrates, reagents, solvents, and products on which a physical or chemical transformation is carried out, and its meaning in the context of the present description and claims is further detailed below.

The term “excipient” means any substance contained in the final pharmaceutical form other than the active ingredient and which generally may not be therapeutically effective by itself. Excipients are essential for the administration of the active substance, as they allow to deliver the drug to the target site. Excipients are commonly referred to as raw materials entering into the composition of a pharmaceutical preparation with the aim of giving a shape, to facilitate administration and preserve the active ingredient. Furthermore, they contribute to characterize the pharmaceutical preparation from the point of view of appearance, stability, biopharmaceutical profile and acceptability by the patient.

X-ray powder diffractometry (XRPD) was used to characterize the amorphous powder obtained by the processes described in this application.

In general, the term “powder” refers to a system in a solid state (as opposed to a liquid or gaseous state) comprising at least two components, wherein one component is dispersed throughout the other component or components.

Amorphous powders generally possess crystal-like short range molecular arrangement (thus generating residual crystallinity broad peaks in the d-spacing between 15-30 Å), but no long range order of molecular packing as found in crystalline solids.

“Stable in the amorphous state upon storage under stressed conditions” in the context of the present invention means that the powder, when stored at a relative humidity of 50% at 25° C. for 24 hours, shows no signs of crystallinity associated to the sodium salts of Valsartan and/or Sacubitril or signs of crystallinity associated to LCZ-696 as judged by the absence of peaks in an X-ray powder diffractogram (XRPD).

By “chemically stable” it is meant that the amorphous powder of the present invention shows no degradation upon storage under stressed conditions, i.e. when stored at 80° C. for 24 hours under vacuum. No degradation means that a HPLC analysis of Valsartan and Sacubitril shows no significant worsening of the purity profile, in terms of formation of new impurities and increase of the content of those already present.

By “physically stable” it is meant that the amorphous powder of the present invention
remains unaltered with respect to its solid state physical parameters upon storage under stressed conditions, e.g. at 50° C. for 24 hours. Furthermore, unaltered with respect to its solid state physical parameters means that after heating to 50° C., the sample maintains the aspect of a granular powder as opposed to, for example, sintered or solid masses.

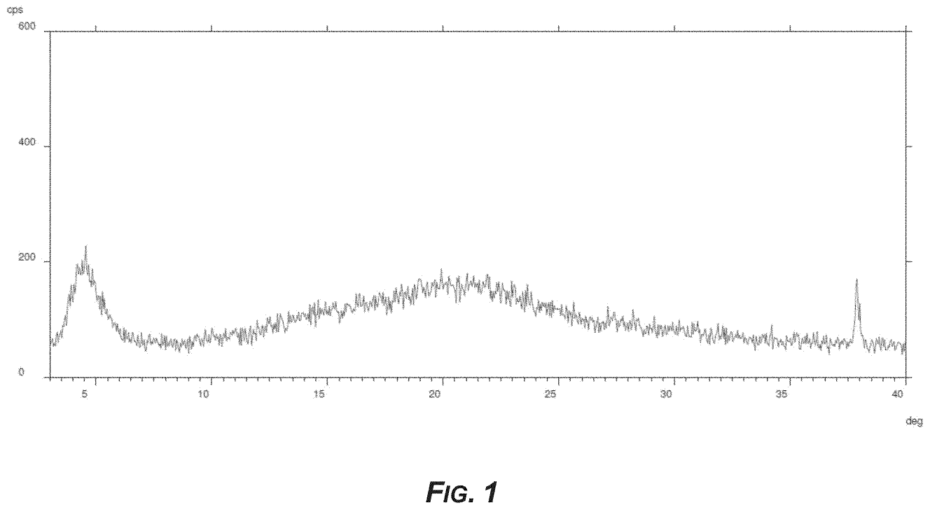

The first aspect of the present invention relates to an amorphous powder comprising a 1:1 stoichiometric mixture of the trisodium salts of Valsartan and Sacubitril having a maximum water content of 4% by weight (preferably lower than 4% by weight). Said powder, which is characterized by a XRPD profile substantially as shown in FIG. 1, may, according to a preferred embodiment of this aspect of the invention, have a maximum water content of 3% by weight, for example lower than 2% by weight. Even more preferably, said amorphous powder comprises a 1:1 stoichiometric mixture of a disodium salt of Valsartan and a monosodium salt of Sacubitril.

The second aspect of the present invention relates to four alternative methods for the preparation of said amorphous powder, which, in the following, will be referred to simply as first to fourth method.

The first method includes steps a) to c).

In step a) LCZ-696 is dissolved in water, or in a mixture comprising water and a water miscible solvent at a temperature preferably between 10 and 30° C. (e.g. between 20 and 25° C.).

According to a preferred operative condition, the organic solvent and the volume ratio between water and the organic solvent are selected so that the pre-freezing temperature of the resulting mixture is not lower than pre-freezing temperature of an aqueous solution containing the same amount of LCZ-696.

The amount of water, or of the mixture comprising water and the water miscible solvent can vary in a very wide range; preferably, the overall volume of liquid may vary between 2 mL and 25 mL per gram of 1:1 stoichiometric mixture of Valsartan and Sacubitril used; even more preferably, said volume ranges between 3 and 15 mL per gram, e.g. 4 mL per gram, of said 1:1 stoichiometric mixture.

Water miscible solvents suitable for the purpose are generally known in the field, such as, for example, C1-C4 alcohols (for example ethanol, methanol or preferably tert-butanol) or C3-C6 ketones (e.g. acetone).

In the subsequent step b) the solution obtained in step a) is freeze dried (lyophilized) according to one of the methods known in the field, comprising, for example, the freezing of a solution followed by a reduction of the pressure to remove the solvent.

Conditions suitable for freezing the solutions of the invention, depending on the solvent chosen to prepare the solution, may entail temperatures ranging between −80° C. and 20° C. (preferably −40° C.) and atmospheric pressure (i.e. about 1 bar). The removal the solvent from the frozen solution may require temperatures generally lower than 20° C. (preferably 0° C.) and pressures preferably ranging between 0.01 and 1 mbar (preferably 0.1 mbar).

In case the water miscible solvent used in step a) is not compatible with the freeze-drying process performed in step b) (for example methanol or acetone), a variant of the first method includes an additional step a′), carried out after step a), in which said solvent is evaporated under reduced pressure. If necessary, the mass obtained after distillation of the organic solvent is diluted with water to obtain a solution, which can be lyophilized.

If the solid resulting from step b) has a water content higher than 4% by weight, the first method includes the next optional step c), consisting in drying said solid. Such a step can be carried out according to any of the procedures generally known in the field, preferably by treating the solid to be dried at temperatures between 30 and 80° C. (e.g. between 35 and 50° C.) under reduced pressure.

The second method of the invention includes steps d) to f).

In step d) LCZ-696 is dissolved in water, a water miscible solvent or a mixture thereof at a temperature preferably between 10 e 30° C. (e.g. between 20 and 25° C.). The amount of water, of water miscible solvent or of the their mixture can vary in a very wide range; preferably, the overall volume of liquid may vary between 2.5 mL and 10 mL per gram of 1:1 stoichiometric mixture of Valsartan and Sacubitril used; even more preferably, the volume is 3 mL per gram of said 1:1 stoichiometric mixture.

Water miscible solvents suitable for the purpose are known in the field, such as, for example, a C1-C4 alcohol (for example ethanol, iso-propanol or preferably methanol or tert-butanol), a C3-C6 ketone (preferably acetone), or an ether (preferably tetrahydrofuran).

In the subsequent step e) the solution obtained in step d) is spray-dried according to one of the methods generally known in the field.

The term “spray-drying” broadly refers to processes involving breaking up liquid mixtures into small droplets (atomization) and rapidly removing solvent from the mixture.

In a typical spray drying apparatus, there is a strong driving force for evaporation of solvent from the droplets, which can be provided by means of a drying gas. Spray drying processes and equipment are described, e.g., in Perry's Chemical Engineer's Handbook, pp. 2054 to 2057 (6th ed. 1984).

By way of example, the typical spray drying apparatus includes a drying chamber, atomizing means for atomizing a solvent-containing feed into the drying chamber, a source of drying gas that flows into the drying chamber to remove solvent from the atomized solvent-containing feed, an outlet for the products of drying, and product collection means located downstream of the drying chamber.

Typically, the product collection means includes a cyclone connected to the drying apparatus. In the cyclone, the particles produced during spray drying are separated from the drying gas and evaporated solvent, allowing the particles to be collected. A filter can also be used to separate and collect the particles produced by spray drying. The drying gas used in the invention can be any suitable gas, preferably air.

The following optional step f) includes, in case the amount of water of the resulting solid is greater than 4% by weight, its drying, according to, for example, the procedure reported above to operate step c).

The third method according to the invention includes steps g) to j).

In step g), a 1:1 stoichiometric mixture of Valsartan and Sacubitril is dispersed in water, a water miscible solvent or a mixture thereof, at a temperature preferably between 10 e 30° C. (e.g. between 20 and 25° C.).

According to a preferred condition, the organic solvent used to disperse the 1:1 stoichiometric mixture of Valsartan and Sacubitril in step g) has a boiling point lower than water.

The amount of water, of water miscible solvent or of the mixture thereof can vary in a very wide range; preferably, the overall volume of liquid may vary between 2 mL and 25 mL per gram of 1:1 stoichiometric mixture of Valsartan and Sacubitril used; even more preferably, the volume ranges between 3 and 15 mL per gram, e.g. 4 mL per gram, of said 1:1 stoichiometric mixture.

Water miscible solvents suitable for the purpose are known and normally used in the field, such as, for example, a C1-C4 alcohol (for example ethanol, iso-propanol or preferably methanol or tert-butanol), a C3-C6 ketone (preferably acetone), or an ether (preferably tetrahydrofuran).

The subsequent step h) comprises the addition of sodium hydroxide in a ratio of 3:1 mole/mole with respect to the 1:1 stoichiometric mixture of Valsartan and Sacubitril.

Sodium hydroxide can be preferably added in the form of an aqueous solution if the previous step was performed in a water miscible solvent alone; alternatively, sodium hydroxide can be added in solid (powder) form. Depending on the way of addition of NaOH, the resulting mixture may be, in turn, either a solution or a wet solid-containing mixture; for this reason, the result of NaOH addition is referred to in this description and in the claims generally as “mass”, which is intended to also include the case that the resulting mixture be a solution. If the preceding steps, and in particular the addition of sodium hydroxide, have been carried out correctly, the resulting mixture should be characterized by the same pH value achieved after dispersing LCZ-696 in the solvent or solvents mixture used to perform steps g) and h). Said standard value may be easily determined by the person skilled in the art by means of a preliminary investigation comprising the dispersion of LCZ-696 into the selected solvent or mixture of solvents and measuring the pH.

By way of example, said value ranges between 9.05 and 9.25, preferably between 9.15 and 9.20, in the case of a 1:1 (v/v) mixture of methanol and water; between 9.40 and 9.80, preferably between 9.60 and 9.65, in the case of a 34:1 (v/v) mixture of acetone and water; and between 8.10 and 8.50, preferably between 8.20 and 8.40, in the case of a 10% (w/w) solution in water.

If, at a control, it is observed that the pH of the mass lies outside said ranges, it is an indication that the stoichiometric ratio between the components of the mixture is not correct; in that case, a variant of the third method of the invention includes an additional step h′), wherein the pH is adjusted within the set range by adding sodium hydroxide (if the pH is lower than the target value) or a solution of the 1:1 stoichiometric mixture of Valsartan and Sacubitril (if the pH is higher than the set value).

Step i) comprises the lyophilization of the solution prepared in step h), after having possibly adjusted the pH of the mass in step h′), using one of the procedures generally known in the field, for example one of those reported above to perform step b).

In case the water miscible solvent used in step g) is not compatible with the freeze-drying process performed in step i), a variant of the process object of the third method includes an additional step h″), carried out after either step h) or h′), in which said solvent (for example methanol or acetone) is evaporated under reduced pressure. If necessary, the mass obtained after distillation of the organic solvent is diluted with water to obtain a solution that can be lyophilized.

The following optional step j), carried out if the amount of water of the resulting solid is higher than 4% by weight, consists in drying the powder, according to, for example, the procedure reported above to operate step c).

The fourth method for the preparation of the 1:1 stoichiometric mixture of the trisodium salts of Valsartan and Sacubitril described above includes steps k) to n).

In step k) a 1:1 stoichiometric mixture of Valsartan and Sacubitril is dispersed in water, a water miscible solvent or a mixture thereof at a temperature preferably between 10 e 30° C. (e.g. between 20 and 25° C.). The amount of water, of water miscible solvent or of the mixture thereof, can vary in a very wide range; preferably, the overall volume of liquid may vary between 2.5 mL and 25 mL per gram of 1:1 stoichiometric mixture of Valsartan and Sacubitril used; even more preferably, the volume is between 10 mL and 15 mL per gram of said 1:1 stoichiometric mixture.

Water miscible solvents suitable for the purpose are known and normally used in the field, such as, for example, a C1-C4 alcohol (for example methanol or ethanol), a C3-C6 ketone (preferably acetone) or an ether (preferably tetrahydrofuran).

The subsequent step l) comprises the addition of sodium hydroxide in a ratio of 3:1 mole/mole with respect to the 1:1 stoichiometric mixture of Valsartan and Sacubitril, according to, e.g., the procedure set out above under point h).

As apparent to any person skilled in the art, the same considerations referred to above under point h) in respect of the pH value of the mass obtained after the addition of sodium hydroxide and to any possible adjustment of this value in order to ensure a complete salification of the available acidic moieties, will likewise apply to step l).

In particular, in case it is observed that the pH of the mass does not correspond to the value achievable by dispersing LCZ-696 in the chosen solvent or mixture of solvents, e.g. the ranges reported above under point h), a variant of the process object of the fourth method includes an additional step l′) wherein said pH is adjusted to the target value by adding sodium hydroxide (if the pH is lower than the set value) or a solution of the 1:1 stoichiometric mixture of Valsartan and Sacubitril (if the pH is higher than the set value).

The next step m) comprises the spray-drying of the solution prepared in step l), after possible adjustment of the pH of the mass in step l′), using one of the procedures generally known in the field, for example one of those reported above to perform step e).

The optional step n), carried out if the amount of water of the resulting solid is higher than 4% by weight, consists in drying the powder according to, for example, the procedure reported above to operate step c).

According to a third aspect of the present invention, the amorphous powder comprising a 1:1 stoichiometric mixture of the trisodium salts of Valsartan and Sacubitril and having a water content of at maximum 4% by weight can be used, in mixture with one or more pharmaceutically acceptable excipients, for the preparation of pharmaceutical compositions useful in the treatment of essential hypertension and/or cardiac failure.

The present invention will be further illustrated by means of the following examples.

XRPD analyses were carried out with an EasyX600 TNX bench-top diffractometer at 25° C., using a CuKα tube (30 kV, 20 mA, λ=1.5408 Å) as the X-ray source, equipped with a strip-detector mod. Dextris. Data collection was made in coupled mode and in theta/theta configuration, with 2theta range increments of 0.018°. Samples were accurately ground and placed in the hollow of a spinning aluminum sampler. The instrument was previously calibrated by means of zinc oxide, then data were collected and elaborated by means of JNyx software. Relative humidity (RH) in the cabin: 28-30%.

The water content of the amorphous powder comprising a 1:1 stoichiometric mixture of the trisodium salts of Valsartan and Sacubitril was determined by Karl Fischer analysis by means of a Mettler-Toledo DL38 automatic titrator, using Combititrant-5 as titrating medium (1 ml correspond to 5 mg of water) and Hydranal Ketosolver as a titration solvent.

Freeze-drying cycle was performed by means of a Martin-Christ GmbH freeze-dryer.

Spray-drying cycle was performed by means of a Buchi B-290 spray-dryer in combination with dehumidifier Buchi B-296.

### Example 1

### Preparation of an Amorphous Powder Comprising a 1:1 Stoichiometric Mixture of the Trisodium Salts of Valsartan and Sacubitril by Means of Freeze-Drying

LCZ-696 (1.0 g) (prepared according to the procedure described in example 1 of international application WO 2007/056546 A1) was dissolved in water (10 mL) under magnetic stirring at 25° C. The obtained solution was freeze-dried according to the following program and ground to obtain an amorphous powder (final water content—as per Karl Fisher titration—9.91% w/w) characterized by an XRPD spectrum as depicted in FIG. 1.

| Step | Time | | Pressure (mbar) | Temperature | |
|---|---|---|---|---|---|
| Pre-freezing | 10 | hours | Atmospheric | −40° | C. |
| | | | pressure | | |
| Primary Drying | 15 | hours | 1 | −20° | C. |
| Step 1 | | | | | |
| Primary Drying | 17 | hours | 1 | −15° | C. |
| Step 2 | | | | | |
| Primary Drying | 1 | hour | 1 | −10° | C. |
| Step 3 | | | | | |
| Primary Drying | 1 | hour | 1 | −5° | C. |
| Step 4 | | | | | |
| Primary Drying | 1 | hour | 1 | 0° | C. |
| Step 5 | | | | | |
| Secondary Drying | 21 | hours | 0.01 | 15° | C. |

### Example 2

### Preparation of an Amorphous Powder Comprising a 1:1 Stoichiometric Mixture of the Trisodium Salts of Valsartan and Sacubitril by Means of Freeze-Drying

LCZ-696 (120.0 g) was dissolved in water (1200 mL) under magnetic stirring at 25° C. The obtained solution was freeze-dried according to the following program and ground to obtain an amorphous powder (final water content—as per Karl Fisher titration—3.70% w/w) characterized by an XRPD spectrum corresponding to the one obtained in Example 1.

| Step | Time | | Pressure (mbar) | Temperature | |
|---|---|---|---|---|---|
| Pre-freezing | 13 | hours | Atmospheric | −35° | C. |
| | | | pressure | | |
| Primary Drying | 17 | hours | 1 | −15° | C. |
| Step 1 | | | | | |
| Primary Drying | 1 | hour | 1 | −10° | C. |
| Step 2 | | | | | |
| Primary Drying | 1 | hour | 1 | −5° | C. |
| Step 3 | | | | | |
| Primary Drying | 1 | hour | 1 | 0° | C. |
| Step 4 | | | | | |
| Secondary Drying | 34 | hours | 0.01 | 15° | C. |

### Example 3

### Preparation of an Amorphous Powder Comprising a 1:1 Stoichiometric Mixture of the Trisodium Salts of Valsartan and Sacubitril by Means of Freeze-Drying

Sacubitril (4.2 g, 10.2 mmol) and Valsartan (4.4 g, 10.2 mmol) were dissolved, under magnetic stirring at 25° C., in a 1:1 (vol/vol) mixture of methanol/water (80 mL). Sodium hydroxide was added (1.2 g, 30.6 mmol) monitoring that the pH of the obtained solution was between 9.15 and 9.20. Methanol was stripped off under reduced pressure and water (30 mL) was added. The obtained solution was freeze-dried according to the program reported in example 1 and ground to obtain an amorphous powder (final water content—as per Karl Fisher titration—8.02% w/w) characterized by an XRPD spectrum corresponding to the one obtained in Example 1.

### Example 4

### Preparation of an Amorphous Powder Comprising a 1:1 Stoichiometric Mixture of the Trisodium Salts of Valsartan and Sacubitril by Means of Freeze-Drying

Sacubitril (30.0 g, 72.9 mmol) and Valsartan (31.7 g, 72.9 mmol) were dissolved, under magnetic stirring at 25° C., in acetone (950 mL). A solution of sodium hydroxide (8.7 g, 21.9 mmol) in water (28 mL) was added monitoring that the pH of the obtained solution was between 9.40 and 9.80. Acetone was stripped off under reduced pressure and water (500 mL) was added. The obtained solution was freeze-dried according to the program reported in example 2 and ground to obtain an amorphous powder (final water content—as per Karl Fisher titration—6.04% w/w) characterized by an XRPD spectrum corresponding to the one obtained in Example 1.

### Example 5

### Preparation of an Amorphous Powder Comprising a 1:1 Stoichiometric Mixture of the Trisodium Salts of Valsartan and Sacubitril by Means of Spray-Drying

LCZ-696 (40.0 g) was dissolved in water (120 mL) under magnetic stirring at 25° C. The resulting solution was filtered and spray-dried by inlet of air at a temperature of 120° C., obtaining a fine powder (final water content—as per Karl Fisher titration—4.10% w/w) characterized by an XRPD spectrum corresponding to the one obtained in Example 1.

### Example 6

### Preparation of an Amorphous Powder Comprising a 1:1 Stoichiometric Mixture of the Trisodium Salts of Valsartan and Sacubitril by Means of Spray-Drying

LCZ-696 (11.0 g) was dissolved in a mixture of acetone (100 mL) and water (5 mL) under magnetic stirring at 35° C. The resulting solution was filtered and spray-dried by inlet of air at a temperature of 64° C., obtaining a fine powder (final water content—as per Karl Fisher titration—5.60% w/w) characterized by an XRPD spectrum corresponding to the one obtained in Example 1.

### Example 7

### Preparation of Amorphous Powders Comprising a 1:1 Stoichiometric Mixture of the Trisodium Salts of Valsartan and Sacubitril Having a Water Content of 0.90% by Weight

The amorphous powders comprising a 1:1 stoichiometric mixture of the trisodium salts of Valsartan and Sacubitril, prepared as described in Example 6, was dried at 40° C. under a reduced pressure of between about 25 and 35 mbar for 12 hours (final water content—as per Karl Fisher titration—3.50% by weight) then at 60° C. under the same vacuum conditions for 10 hours (final water content—as per Karl Fisher titration—0.90% by weight). The solid was brought to room temperature under vacuum and subjected to XRPD analysis, giving rise to a XRPD spectrum corresponding to the one obtained in Example 1.

### Example 8

### Analysis of the Thermal Stability of the Amorphous Powder Comprising a 1:1 Stoichiometric Mixture of the Sodium Salts of Valsartan and Sacubitril Prepared According to the Processes of the Invention

The amorphous powders comprising a 1:1 stoichiometric mixture of the trisodium salts of Valsartan and Sacubitril (10.0 g), prepared as described in Example 7, was maintained at 80° C. under a reduced pressure of between about 25 and 35 mbar for 4 hours. The solid was then brought to room temperature under vacuum and subjected to XRPD analysis, giving rise to a XRPD spectrum corresponding to the one obtained in example 1 (final water content—as per Karl Fisher titration—0.50% w/w).

### Example 9

### Analysis of the Moisture Stability of the Amorphous Powder Comprising a 1:1 Stoichiometric Mixture of the Sodium Salts of Valsartan and Sacubitril Prepared According to the Processes of the Invention

The amorphous powder comprising a 1:1 stoichiometric mixture of the trisodium salts of Valsartan and Sacubitril (10.0 g), prepared as described in Example 4, was placed at 20-25° C. in a hydration chamber containing a saturated solution of potassium carbonate for 24 hours (50%±5% RH). The solid was then subjected to XRPD analysis, giving rise to a XRPD spectrum corresponding to the one obtained in example 1 (final water content—as per Karl Fisher titration—9.10% by weight).

### Example 10

### Analysis of the Physical Stability of the Amorphous Powder Comprising a 1:1 Stoichiometric Mixture of the Trisodium Salts of Valsartan and Sacubitril as a Function of its Water Content

The physical stability of the amorphous powder comprising a 1:1 stoichiometric mixture of the trisodium salts of Valsartan and Sacubitril as a function of its water content was determined by storing samples of the powder in a closed vial for 24 hours at different temperatures. The results of these tests are summarized in Table 1.

TABLE 1

| Water | | | | |
|---|---|---|---|---|
| Content | Temperature | | | |
| (K.F.) | 40° C. | 50° C. | 60° C. | 80° C. |
| 1.5% | Powder | Powder | Powder | Powder |
| 3% | Powder | Powder | Powder | Powder |
| 4% | Powder | Powder | Sintered | Sintered |
| | | | grainy mass | grainy mass |
| 5% | Powder | Sintered | Solid Mass | Solid Mass |
| | | grainy mass | | |
| 7% | Powder | Solid Mass | Solid Mass | Solid Mass |
| | with lumps | | | |

### Example 11 (Comparative)

For purposes of comparison with the results of the present invention, the procedure reported in Example 1 of WO 2007/056546 A1 (referred to below as WO '546) has been accurately repeated but on a bigger scale in order to reduce, as much as possible, the experimental error associated with measures.

Sacubitril (19.0 g, 46.2 mmol) and Valsartan (19.5 g, 44.8 mmol) were dissolved in acetone (1900 mL) under magnetic stirring at 25° C. A solution of sodium hydroxide (5.3 g, 132.2 mmol) in water (333 mL) was added to the former solution maintaining the resulting mixture under stirring up to obtaining a clear solution (pH=9.44). The solution was evaporated at 35° C. for 9 hours under reduced pressure (between about 15 and 25 mbar) to yield 44.1 g of a glassy solid characterized by an XRPD spectrum compatible with an amorphous powder and having a water content of 5.7% by weight.

As a result of this comparative test, the present inventors have found that the procedure described in WO '546 does not actually lead to a 1:1 stoichiometric mixture of the trisodium salts of Valsartan and Sacubitril characterized by containing an amount of water of at maximum 4% by weight (object of the present application), but rather to:
1) a glassy solid comprising a non-stoichiometric ratio of Valsartan and Sacubitril. In this respect, it is worth noting that according to example 1 of WO '546, 0.96 mmoles of Sacubitril free acid (taking into account the indicated degree of purity) and 0.94 mmoles of Valsartan free acid are dissolved in acetone, thus leading, after evaporation at 35° C., to a solid containing a non-stoichiometric ratio of Valsartan and Sacubitril;
2) a glassy solid comprising a mixture of Valsartan, Sacubitril and the sodium salts thereof. In particular, upon treating the acetone solution containing a non-stoichiometric ratio of Valsartan and Sacubitril with a solution of sodium hydroxide in water, the use of only 2.77 mmoles of sodium hydroxide (instead of the required 2.85 mmoles) results in an incomplete salification of the available acidic functions. As a further evidence of the latter observation, the present inventors have experimentally observed a discrepancy between the pH of the solution prepared according to WO '546 (i.e. 9.44) and that measured after dissolving LCZ-696 in the same water/acetone mixture (namely 9.62). Said inconsistency is compatible with the presence, in the solution to be evaporated and in the glassy solid resulting therefrom, of partially not salified Valsartan and/or Sacubitril free acids;
3) a glassy solid having a non-specified water content. The solid form obtained in the evaporation process mentioned in WO '546 may or may not be, depending upon the operative conditions used, the amorphous powder object of the present invention. In particular, the disclosed conditions do not specify any evaporation conditions apart from the temperature to which said operation should be carried out; nor the authors of WO '546 report, for example, the water content of the resulting glassy solid, the time of the treatment and the level of vacuum to be applied during the evaporation. Hence, the process mentioned in WO '546 is not sufficiently defined to provide a direct disclosure of an amorphous powder containing a specified amount of water since, depending on the choice of the different evaporation conditions, glassy solids with different water contents might be obtained.

### COMMENTS TO THE RESULTS

As clearly derivable from Table 1 above, all the amorphous powders prepared according to the methods of the invention, comprising a 1:1 stoichiometric mixture of the trisodium salts of Valsartan and Sacubitril and having a water content of at maximum 4% by weight, have an improved stability to high temperatures since they do not convert into a sintered or a solid mass when heated to 50° C. for 24 hours. Moreover, it is worth noting that, when the water content in the samples is lower than 4%, e.g. 3%, the resulting powder is physically stable even if subjected to heating to 60 or 80° C.

On the contrary, the product obtained following the procedure of WO '546, does not provide comparably good results. In particular, the methods of the present invention allow to consistently obtain a solid with desired properties as to pH, stoichiometry of the composition and water content; on the other hand, the procedure described in WO '546 does not afford the same control over the final result, giving rise to a non-negligible variability of the stoichiometry of the Valsartan/Sacubitril ratio, or of the water content of the powder; this latter parameter, as shown by the experiments of the present inventors, in turn results in a remarkable variability (and thus lack of control) of the physical stability of the product.

What is claimed is:

1. A method for producing an amorphous powder comprising a 1:1 stoichiometric mixture of the trisodium salts of Valsartan and Sacubitril and having a water content of at maximum 4% by weight, wherein the method is selected from the group consisting of Methods A, B, C, and D, and further wherein:
Method A comprises:
(a) dissolving the supramolecular complex trisodium [3-((1S,3R)-1-biphenyl-4-yl-methyl-3-ethoxycarbonyl-1-butylcarbamoyl)propionate-(S)-3′-methyl-2′-(pentanoyl{2″-(tetrazol-5-ylate)biphenyl-4′-yl-methyl}amino)butyrate]-hemi-pentahydrate (LCZ-696) in water or in a mixture comprising water and a water miscible solvent;
(b) freeze-drying the solution obtained in step (a); and
(c) optionally, if the resulting solid contains an amount of water greater than 4% by weight, drying it;
Method B comprises:
(d) dissolving the supramolecular complex trisodium [3-((1S,3R)-1-biphenyl-4-yl-methyl-3-ethoxycarbonyl-1-butylcarbamoyl)propionate-(S)-3′-methyl-2′-(pentanoyl{2″-(tetrazol-5-ylate)biphenyl-4′-yl-methyl}amino)butyrate]-hemi-pentahydrate (LCZ-696) in water, a water miscible solvent or a mixture thereof;
(e) spray-drying the solution obtained in step (d); and
(f) optionally, if the resulting solid contains an amount of water greater than 4% by weight, drying it;
Method C comprises:
(g) dispersing a 1:1 stoichiometric mixture of Valsartan and Sacubitril in water, a water miscible solvent or a mixture thereof;
(h) adding sodium hydroxide in a ratio of 3:1 mole/mole with respect to the 1:1 stoichiometric mixture of Valsartan and Sacubitril;
(i) freeze-drying the solution obtained in step (h); and
(j) optionally, if the resulting solid contains an amount of water greater than 4% by weight, drying it;
Method D comprises:
(k) dispersing a 1:1 stoichiometric mixture of Valsartan and Sacubitril in water, a water miscible solvent or a mixture thereof;
(l) adding sodium hydroxide in a ratio of 3:1 mole/mole with respect to the 1:1 stoichiometric mixture of Valsartan and Sacubitril;
(m) spray-drying the solution obtained in step (l); and
(n) optionally, if the resulting solid contains an amount of water greater than 4% by weight, drying it.

2. The method of claim 1, wherein Method A further comprises step (a′), and wherein step (a′) is performed between steps (a) and (b) and comprises distilling off the water miscible solvent and, optionally, diluting the mass with water to obtain a solution.

3. The method of claim 1, wherein when in step (g) of Method B a water miscible solvent or a mixture thereof with water is used, said solvent is selected from the group consisting of methanol, tert-butanol, and acetone.

4. The method of claim 1, wherein Method C further comprises step (h′), and wherein step (h′) is performed between steps (h) and (i) and comprises adjusting the pH of the mass obtained in step (h) to the value resulting from the dispersion of LCZ-696 in the solvent or solvent mixture used to perform steps (g) and (h).

5. The method of claim 1, wherein Method C further comprises a step (h″), wherein step (h″) is performed after step (h) and comprises distilling off the water miscible solvent and, optionally, diluting the mass with water to obtain a solution.

6. The method of claim 1, wherein the water miscible solvent used in step (k) of Method C is acetone.

7. The method of claim 1, wherein Method D further comprises step (l′), and wherein step (l′) is performed between steps (l) and (m) and comprises adjusting the pH of the mass obtained in step (l) to the value resulting from dispersing LCZ-696 in the solvent or solvent mixture used to perform steps (k) and (l).

8. The method of claim 1, wherein Method C further comprises:
(h′) adjusting the pH of the mass obtained in step (h) to the value resulting from dispersing LCZ-696 in the solvent or solvent mixture used to perform steps (g) and (h); and
(h″) distilling off the water miscible solvent and, optionally, diluting the mass with water to obtain a solution;
and further wherein adjusting step (h′) is performed between steps (h) and (i) and distilling step (h″) is performed after step (h′).
